(19) United States
(12) Patent Application Publication
(10) Pub. No.: US 2007/0016057 A1
(43) Pub. Date: Jan. 18, 2007

(54) Medical device having gripping layer

(76) Inventors: Anthony Dallago, Chandler, AZ (US); Jeff Esker, Mesa, AZ (US)

Correspondence Address: THE SMALL PATENT LAW GROUP LLP, 611 OLIVE STREET, SUITE 1611, ST. LOUIS, MO, 63101, US

(21) Appl. No.: 11/182,129

(22) Filed: Jul. 15, 2005

(51) Int. Cl. A61B 8/14

(52) U.S. Cl. 600/459

(57) ABSTRACT

A medical device having a gripping layer and a method of manufacturing a medical device are provided. A medical device housing of the medical device includes a handle extending along a length of the medical device housing. The handle has a proximal end configured to be held proximate an area of interest of a patient and a distal end configured to be remote from the area of interest. The handle includes an intermediate portion between the proximal and distal ends. The medical device housing further includes a gripping layer provided over at least a portion of the intermediate portion. The gripping layer is positioned along the handle to be held by a user, with the gripping layer made of a softer material than the handle.

### BACKGROUND OF THE INVENTION

This invention relates generally to medical imaging systems, and more particularly, to medical devices for the medical imaging systems.

Medical imaging systems, and in particular, ultrasound systems typically include ultrasound scanning devices, such as, ultrasound probes having different control components and transducers that allow for performing various different ultrasound scans (e.g., different imaging of a volume or body). These ultrasound probes may include control components within different portions of the probe, including, for example, the probe handle. These control components within the probe allow for controlling operation of the probe by an ultrasound system, for example, to operate in different modes, such as, amplitude mode (A-mode), brightness mode (B-1 mode), power Doppler mode, color imaging mode, among others.

These ultrasound probes are typically constructed having a one piece or two piece handle design and formed with a hard plastic material to protect the components within the handle. The ultrasound probe also typically includes a head shell or nose piece connected to an end of the ultrasound probe and which is also formed of a hard plastic. The hard plastic material for each of the component parts of the handle and head shell is the same.

Because the probe handle is constructed of a hard plastic material, the design of the handle, including the contours have to be formed initially in the design and manufacturing processes. Any change would require a change to the design and/or manufacturing process. This hard plastic handle design provides limited flexibility, for example, to form contours or gripping portions on the handle for users having different size hands. Further, the hard plastic material is sometimes difficult to grip by a user when performing an examination, for example, while scanning a patient and moving the probe along a surface.

### BRIEF DESCRIPTION OF THE INVENTION

In one embodiment, a medical device housing is provided that includes a handle extending along a length of the medical device housing. The handle has a proximal end configured to be held proximate an area of interest of a patient and a distal end configured to be remote from the area of interest. The handle includes an intermediate portion between the proximal and distal ends. The medical device housing further includes a gripping layer provided over at least a portion of the intermediate portion. The gripping layer is positioned along the handle to be held by a user, with the gripping layer made of a softer material than the handle.

In another embodiment, a medical device is provided that includes a housing having a handle extending along a length of the housing, with the handle having a first end and a second end. The housing is formed of a first material. The medical device further includes an intermediate portion between the first and second ends and a gripping layer provided over at least a portion of the intermediate portion, with the gripping layer formed of a second material softer than the first material.

In yet another embodiment, a method of manufacturing a medical device handle is provided that includes forming a handle of a first material. The handle extends along a length thereof and has a proximal end configured to be held proximate an area of interest of a patient and a distal end configured to be remote from the area of interest. The handle has an intermediate portion between the proximal and distal ends. The method further includes forming a second material onto a gripping layer over at least a portion of the intermediate portion. The gripping layer is positioned along the handle to be held by a user. The second material is made of a softer material than the first material.

### BRIEF DESCRIPTION OF THE DRAWINGS

FIG. 1 is a block diagram of an ultrasound system in accordance with an exemplary embodiment of the present invention.

FIG. 2 is a block diagram of an ultrasound system in accordance with another exemplary embodiment of the present invention.

FIG. 3 is a perspective view of an image of an object acquired by the systems of FIGS. 1 and 2 in accordance with an exemplary embodiment of the present invention.

FIG. 4 is a plan view of an exemplary ultrasound probe constructed in accordance with an embodiment of the invention.

FIG. 5 is a plan view of an exemplary ultrasound probe constructed in accordance with another embodiment of the invention.

FIG. 6 is a plan view of an exemplary ultrasound probe constructed in accordance with another embodiment of the invention.

FIG. 7 is a plan view of the ultrasound probe of FIG. 4 without a gripping layer.

### DETAILED DESCRIPTION OF THE INVENTION

Exemplary embodiments of medical devices, and more particularly, ultrasound probes having a gripping portion are described in detail below. In particular, a detailed description of exemplary ultrasound systems in connection with which the probes may be used is first provided followed by a detailed description of various embodiments of the ultrasound probes.

It should be noted that although the various embodiments of probes described herein are constructed as ultrasound probes, the various embodiments of the invention are not limited to ultrasound probes. For example, the various embodiments may be implemented with any type of medical device, for example, a biopsy probe, a dental instrument, a surgical instrument, a patient monitoring device and a diagnostic probe, among others.

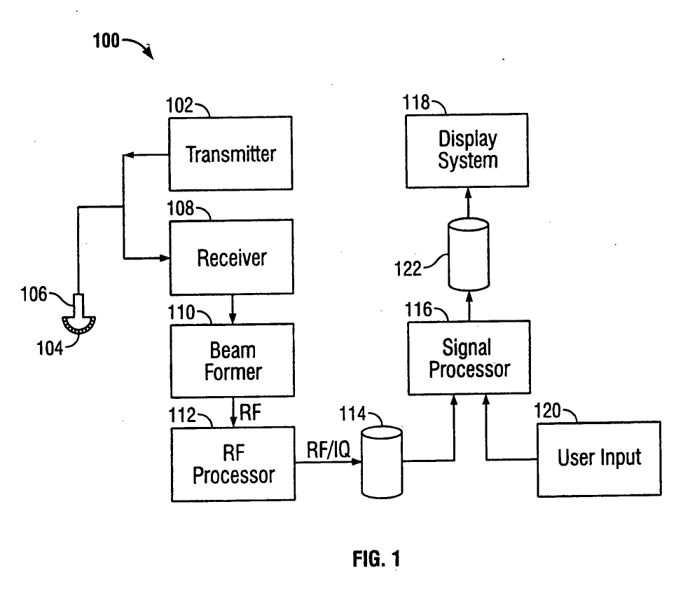

FIG. 1 illustrates a block diagram of an exemplary embodiment of an ultrasound system 100 that may be used, for example, to acquire and process ultrasonic images. The ultrasound system 100 includes a transmitter 102 that drives an array of elements 104 (e.g., piezoelectric crystals) within or formed as part of a transducer 106 to emit pulsed ultrasonic signals into a body or volume. A variety of geometries may be used and one or more transducers 106 may be provided as part of a probe as described in more detail below. The pulsed ultrasonic signals are back-scattered from density interfaces and/or structures, for example, in a body, like blood cells or muscular tissue, to produce echoes that return to the elements 104. The echoes are received by a receiver 108 and provided to a beamformer 110. The beamformer performs beamforming on the received echoes and outputs an RF signal. The RF signal is then processed by an RF processor 112. The RF processor 112 may include a complex demodulator (not shown) that demodulates the RF signal to form IQ data pairs representative of the echo signals. The RF or IQ signal data then may be routed directly to an RF/IQ buffer 114 for storage (e.g., temporary storage).

The ultrasound system 100 also includes a signal processor 116 to process the acquired ultrasound information (i.e., RF signal data or IQ data pairs) and generate frames of ultrasound information for display on a display system 118. The signal processor 116 is adapted to perform one or more processing operations according to a plurality of selectable ultrasound modalities on the acquired ultrasound information. Acquired ultrasound information may be processed in real-time during a scanning session as the echo signals are received. Additionally or alternatively, the ultrasound information may be stored temporarily in the RF/IQ buffer 114 during a scanning session and processed in less than real-time in a live or off-line operation.

The ultrasound system 100 may continuously acquire ultrasound information at a frame rate that exceeds fifty frames per second, which is the approximate perception rate of the human eye. The acquired ultrasound information is displayed on the display system 118 at a slower frame-rate. An image buffer 122 may be included for storing processed frames of acquired ultrasound information that are not scheduled to be displayed immediately. In an exemplary embodiment, the image buffer 122 is of sufficient capacity to store at least several seconds of frames of ultrasound information. The frames of ultrasound information may be stored in a manner to facilitate retrieval thereof according to their order or time of acquisition. The image buffer 122 may comprise any known data storage medium.

A user input device 120 may be used to control operation of the ultrasound system 100. The user input device 120 may be any suitable device and/or user interface for receiving user inputs to control, for example, the type of scan or type of transducer to be used in a scan.

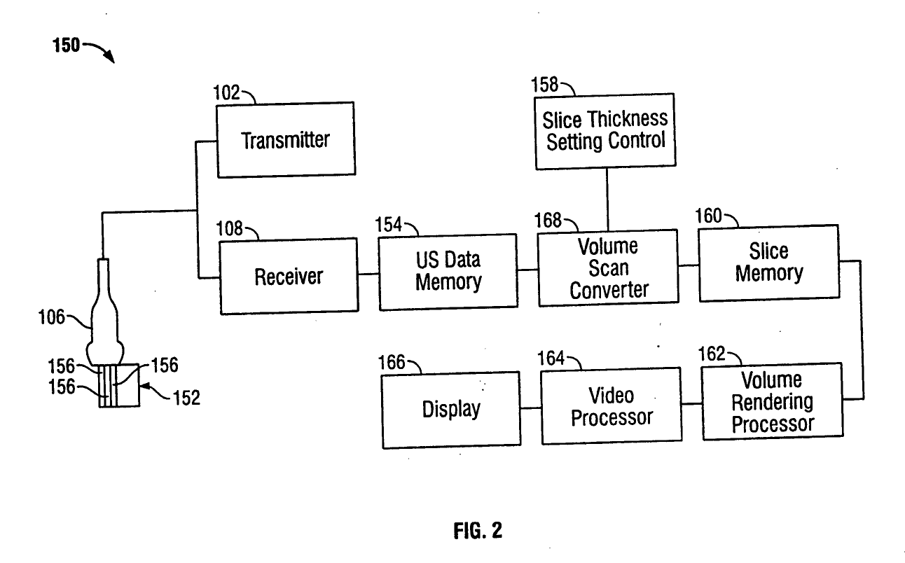

FIG. 2 illustrates a block diagram of another exemplary embodiment of an ultrasound system 150 that may be used, for example, to acquire and process ultrasonic images. The ultrasound system 150 includes the transducer 106 in communication with the transmitter 102 and receiver 108. The transducer 106 transmits ultrasonic pulses and receives echoes from structures inside a scanned ultrasound volume 152. A memory 154 stores ultrasound data from the receiver 108 derived from the scanned ultrasound volume 152. The scanned ultrasound volume 152 may be obtained by various techniques, including, for example, 3D scanning, real-time 3D imaging, volume scanning, scanning with transducers having positioning sensors, freehand scanning using a Voxel correlation technique, 2D scanning or scanning with a matrix of array transducers, among others.

The transducer 106 is moved, such as along a linear or arcuate path, while scanning a region of interest (ROI). At each linear or arcuate position, the transducer 106 obtains a plurality of scan planes 156. The scan planes 156 are collected for a thickness, such as from a group or set of adjacent scan planes 156. The scan planes 156 are stored in the memory 154, and then provided to a volume scan converter 168. In some exemplary embodiments, the transducer 106 may obtain lines instead of the scan planes 156, with the memory 154 storing lines obtained by the transducer 106 rather than the scan planes 156. The volume scan converter 168 receives a slice thickness setting from a slice thickness setting control 158, which identifies the thickness of a slice to be created from the scan planes 156. The volume scan converter 168 creates a data slice from multiple adjacent scan planes 156. The number of adjacent scan planes 156 that are obtained to form each data slice is dependent upon the thickness selected by the slice thickness setting control 158. The data slice is stored in a slice memory 160 and accessed by a volume rendering processor 162. The volume rendering processor 162 performs volume rendering upon the data slice. The output of the volume rendering processor 162 is provided to a video processor 164 that processes the volume rendered data slice for display on a display 166.

It should be noted that the position of each echo signal sample (Voxel) is defined in terms of geometrical accuracy (i.e., the distance from one Voxel to the next) and one or more ultrasonic responses (and derived values from the ultrasonic response). Suitable ultrasonic responses include gray scale values, color flow values, and angio or power Doppler information.

It should be noted that the ultrasound systems 100 and 150 may include additional or different components. For example, the ultrasound system 150 may include a user interface or user input 120 (shown in FIG. 1) to control the operation of the ultrasound system 150, including, to control the input of patient data, scan parameters, a change of scan mode, and the like.

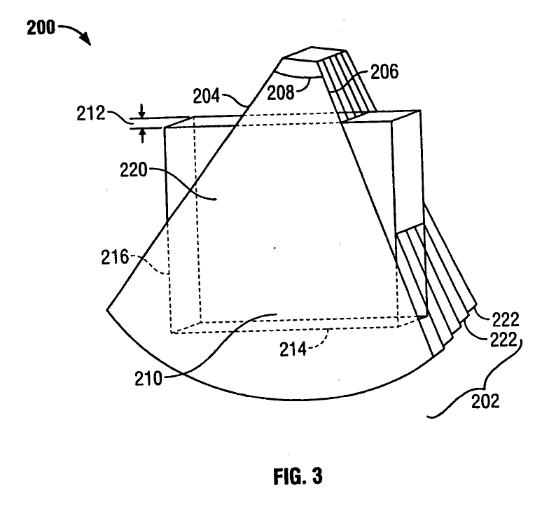

FIG. 3 illustrates an exemplary image of an object 200 that may be acquired by the ultrasound systems 100 and 150. The object 200 includes a volume 202 defined by a plurality of sector shaped cross-sections with radial borders 204 and 206 diverging from one another at an angle 208. The transducer 106 (shown in FIGS. 1 and 2) electronically focuses and directs ultrasound firings longitudinally to scan along adjacent scan lines in each scan plane 156 (shown in FIG. 2) and electronically or mechanically focuses and directs ultrasound firings laterally to scan adjacent scan planes 156. The scan planes 156 obtained by the transducer 106, and as illustrated in FIG. 1, are stored in the memory 154 and are scan converted from spherical to Cartesian coordinates by the volume scan converter 168. A volume comprising multiple scan planes 156 is output from the volume scan converter 168 and stored in the slice memory 160 as a rendering region 210. The rendering region 210 in the slice memory 160 is formed from multiple adjacent scan planes 156.

The rendering region 210 may be defined in size by an operator using a user interface or input to have a slice thickness 212, width 214 and height 216. The volume scan converter 168 (shown in FIG. 2) may be controlled by the slice thickness setting control 158 (shown in FIG. 2) to adjust the thickness parameter of the slice to form a rendering region 210 of the desired thickness. The rendering region 210 defines the portion of the scanned ultrasound volume 152 that is volume rendered. The volume rendering processor 162 accesses the slice memory 160 and renders along the slice thickness 212 of the rendering region 210.

Referring now to FIGS. 1 and 2, during operation, a slice having a pre-defined, substantially constant thickness (also referred to as the rendering region 210) is determined by the slice thickness setting control 158 and is processed in the volume scan converter 168. The echo data representing the rendering region 210 (shown in FIG. 3) may be stored in the slice memory 160. Predefined thicknesses between about 2 mm and about 20 mm are typical, however, thicknesses less than about 2 mm or greater than about 20 mm may also be suitable depending on the application and the size of the area to be scanned. The slice thickness setting control 158 may include a control member, such as a rotatable knob with discrete or continuous thickness settings.

The volume rendering processor 162 projects the rendering region 210 onto an image portion 220 of an image plane(s) 222 (shown in FIG. 3). Following processing in the volume rendering processor 162, pixel data in the image portion 220 may be processed by the video processor 164 and then displayed on the display 166. The rendering region 210 may be located at any position and oriented at any direction within the volume 202. In some situations, depending on the size of the region being scanned, it may be advantageous for the rendering region 210 to be only a small portion of the volume 202.

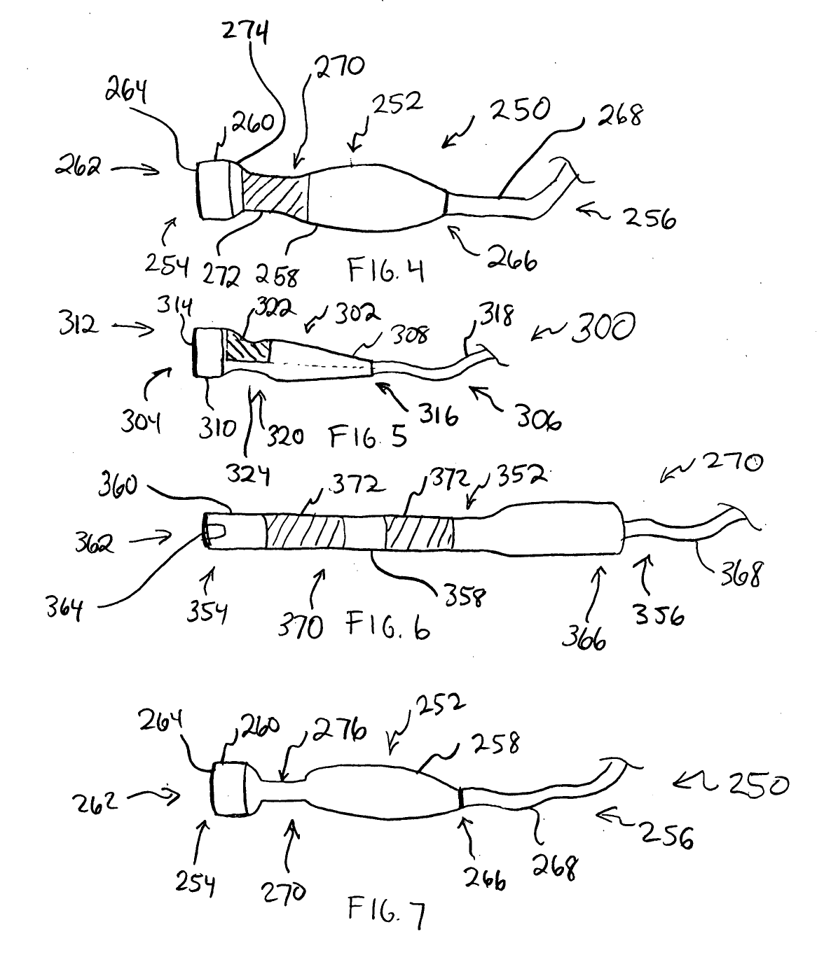

FIGS. 4 through 6 illustrate an ultrasound probes 250, 300 and 350, respectively, constructed in accordance with exemplary embodiments of the invention. The ultrasound probe 250 shown in FIG. 4 generally includes a housing 252 having a scan portion 254 and a connection portion 256. The housing 252 may be formed of a single piece construction or a two piece construction (e.g., two half shells), as is known, and generally defines a handle 258 and includes a nose piece 260 on a first end 262 of the handle 258 at the scan portion 254. The first end 262 includes a lens 264 configured to, for example, contact or be held proximate an area of interest of a patient to be scanned with the ultrasound probe 250. The first end 262 typically includes therein the scanning components of the ultrasound probe 250, including, for example, a transducer assembly (not shown) as is known. The connection portion 256 is provided at a second end 266 of the handle 258 and may include a flex relief with a system cable 268 extending therefrom and configured for connection to an ultrasound system, for example, the ultrasound system 100 or 150 (shown in FIGS. 1 and 2).

An intermediate portion 270 is provided between the first end 262 and the second end 266, namely, between a proximal end and a distal end. The intermediate portion 270 includes a gripping layer 272 configured for holding by a user of the ultrasound probe 250. The gripping layer 272 is formed of a material softer than the material of the housing 252, and more particularly, the handle 258. For example, in an exemplary embodiment, the handle 258 extends along the length of the ultrasound probe 250 and is formed of a polymer, such as a plastic, having a hardness of greater than 80 durometers. The handle 258 may be formed, for example, of different types of hard plastic, such as Valox 357. The gripping layer 272, in an exemplary embodiment, is formed of a polymer softer than the polymer of the handle 258, such as an elastomer having a hardness of between about 60 durometers and 80 durometers. The gripping layer 272 may be formed, for example, of a rubber or silicone composite material. Additionally, the lens 264 is generally also formed from a polymer softer than the polymer of the handle 258, and in an exemplary embodiment, has a hardness of between about 50 durometers and 60 durometers.

The gripping layer 272 may be comolded or overmolded on the handle 258 using any known and suitable comolding or overmolding process. For example, as shown in FIG. 7, the handle 258 may be formed having an indented portion 276 that is overmolded or comolded with the gripping layer 272. Thus, the housing 252 is formed of a polymer and the gripping layer 272 is formed of a polymer softer than the polymer of the housing 252 and is comolded or overmolded to the intermediate portion 270 of the handle 258. The gripping layer 272 may extend onto and cover, for example, a portion of the nose piece 260, namely, to overlap the nose piece 260 and extend beyond the indented portion 276.

Additionally, the ultrasound probe 250 may be formed such that the gripping layer 272 is configured to define a gripping portion or contact area for a user, such as, a stenographer. For example, the handle 258 may be contoured such that the intermediate portion 270 has a smaller outside diameter than the outside diameter of the handle 258 at the first end 262. The gripping layer 272 also may extend along only a portion of the handle 258, or may extend along the entire length of the handle 258. The handle 258 also may include, for example, a neck portion 274 to provide leverage for the user and/or to facilitate loading provided by the user when using the ultrasound probe 250. The neck portion 274 may be formed, for example, by an indented region of the handle 258.

As shown in FIG. 5, in another exemplary embodiment, an ultrasound probe 300 generally includes a housing 302 having a scan portion 304 and a connection portion 306. The housing 302 may be formed of a single piece construction or a two piece construction, as is known, and generally defines a handle 308 and includes a nose piece 310 on a first end 312 of the handle 308 at the scan portion 304. The first end 312 includes a lens 314 configured to, for example, contact or be held proximate an area of interest of a patient to be scanned with the ultrasound probe 300. The first end 312 typically includes therein the scanning components of the ultrasound probe 300, including, for example, a transducer assembly (not shown) as is known. The connection portion 306 is provided at a second end 316 of the handle 308 and may include a flex relief with a system cable 318 extending therefrom and configured for connection to an ultrasound system, for example, the ultrasound system 100 or 150 (shown in FIGS. 1 and 2).

An intermediate portion 320 is provided between the first end 312 and the second end 316, namely, between a proximal end and a distal end. The intermediate portion 320 includes a gripping layer 322 configured for holding by a user of the ultrasound probe 300. The gripping layer 322 is formed of a material softer than the material of the housing 302, and more particularly, the handle 308, and in this embodiment, surrounds only a portion of the diameter of the handle 308. In an exemplary embodiment, the handle 308 extends along the length of the ultrasound probe 300 and is formed of a polymer, such as a plastic, having a hardness of greater than 80 durometers. The handle 308 may be formed, for example, of different types of hard plastic, such as Valox 357. The gripping layer 322, in an exemplary embodiment, is formed of a polymer softer than the polymer of the handle 308, such as an elastomer having a hardness of between about 60 durometers and 80 durometers. The gripping layer 322 may be formed, for example, of a rubber or silicone composite material. Additionally, the lens 314 is generally also formed from a polymer softer than the polymer of the handle 308, and in an exemplary embodiment, has a hardness of between about 50 durometers and 60 durometers.

The gripping layer 322 may be comolded or overmolded on the handle 308 using any known and suitable comolding or overmolding process. Thus, the housing 302 is formed of a polymer and the gripping layer 322 is formed of a polymer softer than the polymer of the housing 302 and is comolded or overmolded to the intermediate portion 320 of the handle 308. The gripping layer 322 may extend onto and cover, for example, a portion of the nose piece 310, namely, to overlap the nose piece 310.

Additionally, the ultrasound probe 300 may be formed such that the gripping layer 322 is configured to define a gripping portion or contact area for a user, such as, a stenographer. For example, the handle 258 may be contoured such that the intermediate portion 320 has a smaller outside diameter than the outside diameter of the handle 308 at the first end 312. The gripping layer 322 also may extend along only a portion of the handle 308, or may extend along the entire length of the handle 308. The handle 258 also may include, for example, an indented portion 324 to provide leverage for the user and/or to facilitate loading provided by the user when using the ultrasound probe 250.

As shown in FIG. 6, in another exemplary embodiment, an ultrasound probe 350 generally includes a housing 352 having a scan portion 354 and a connection portion 356. The housing 352 may be formed of a single piece construction or a two piece construction, as is known, and generally defines a handle 358 and includes a nose piece 360 on a first end 362 of the handle 358 at the scan portion 354. The first end 362 includes a lens 364 configured to, for example, contact or be held proximate an area of interest of a patient to be scanned with the ultrasound probe 350. The first end 362 typically includes therein the scanning components of the ultrasound probe 350, including, for example, a transducer assembly (not shown) as is known. The connection portion 356 is provided at a second end 366 of the handle 358 and may include a flex relief with a system cable 368 extending therefrom and configured for connection to an ultrasound system, for example, the ultrasound system 100 or 150 (shown in FIGS. 1 and 2).

An intermediate portion 370 is provided between the first end 362 and the second end 366, namely, between a proximal end and a distal end. The intermediate portion 370, in this embodiment, includes two separate gripping layers 372 configured for holding by a user of the ultrasound probe 350. The gripping layers 372 are formed of a material softer than the material of the housing 352, and more particularly, the handle 358. For example, in an exemplary embodiment, the handle 358 extends along the length of the ultrasound probe 350 and is formed of a polymer, such as a plastic, having a hardness of greater than 80 durometers. The handle 358 may be formed, for example, of different types of hard plastic, such as Valox 357. The gripping layers 372, in an exemplary embodiment, are formed of a polymer softer than the polymer of the handle 358, such as an elastomer having a hardness of between about 60 durometers and 80 durometers. The gripping layers 372 may be formed, for example, of a rubber or silicone composite material. Additionally, the lens 364 is generally also formed from a polymer softer than the polymer of the handle 358, and in an exemplary embodiment, has a hardness of between about 50 durometers and 60 durometers.

The gripping layers 372 may be comolded or overmolded on the handle 358 using any known and suitable comolding or overmolding process. Thus, the housing 352 is formed of a polymer and the gripping layers 372 are formed of a polymer softer than the polymer of the housing 352 and are comolded or overmolded to an intermediate portion 370 of the handle 358. One of the gripping layers 372 may extend onto and cover, for example, a portion of the nose piece 360, namely, to overlap the nose piece 360.

Additionally, the ultrasound probe 350 may be formed such that the gripping layers 372 are configured to define gripping portions or contact areas for a user, such as, a stenographer. For example, the handle 358 may be configured such the gripping layers 372 are provided thereto spaced apart for a user to be able to hold the ultrasound probe 350 with one or two hands.

Thus, various embodiments of the present invention provide an ultrasound probe having a gripping layer that is formed of a material softer than the material of a handle of the ultrasound probe. Essentially, a gripping layer is comolded or overmolded to the handle of the ultrasound probe.

It should be noted that the hardness of the materials for the component parts of the various embodiments of the invention may be modified to be harder or softer as desired or needed. Further, the type and color of the materials may be modified as desired or needed.

While the invention has been described in terms of very specific embodiments, those skilled in the art will recognize that the invention can be practiced with modification within the spirit and scope of the claims.

What is claimed is:

1. A medical device housing comprising:
a handle extending along a length, said handle having a proximal end configured to be held proximate an area of interest of a patient and a distal end configured to be remote from the area of interest, said handle having an intermediate portion between the proximal and distal ends; and
a gripping layer provided over at least a portion of said intermediate portion, said gripping layer being positioned along said handle to be held by a user, said gripping layer made of a softer material than said handle.

2. The medical device housing of claim 1, wherein said handle comprises a material having a hardness of at least 80 durometers and said gripping layer comprises a material having a hardness of less than 80 durometers.

3. The medical device housing of claim 1, wherein said gripping layer comprises a material having a hardness of between 60 and 80 durometers.

4. The medical device housing of claim 1, wherein said gripping layer is overmolded onto said intermediate portion.

5. The medical device housing of claim 1, wherein said gripping layer is comolded with said handle.

6. The medical device housing of claim 1, wherein said intermediate portion of said handle entirely laterally surrounds a chamber within said handle, said gripping layer being bonded to an exterior surface of said intermediate portion.

7. The medical device housing of claim 1, wherein said handle is engaged to a nose piece provided at said proximal end of said handle, said gripping layer abutting against without overlapping said nose piece.

8. The medical device housing of claim 1, wherein said handle is contoured such that said intermediate portion has a smaller outer perimeter than an outer perimeter of said handle at said proximal end.

9. The medical device housing of claim 1, wherein said intermediate portion of said handle is at least partially indented to form a neck area proximate said proximal end of said handle, said gripping layer at least partially overlapping said neck area where said handle is held by the user.

10. The medical device housing of claim 1, wherein said handle comprises two half shells joined to one another along the length of said handle, said gripping layer overlapping at least one of said half shells.

11. The medical device housing of claim 1, wherein said handle comprises one of an ultrasound probe handle, a biopsy probe handle, a dental instrument handle, a surgical instrument handle, a patient monitoring device handle and a diagnostic instrument handle.

12. A medical device comprising:
a housing including a handle extending along a length of the housing, the handle having a first end and a second end, the housing formed of a first material;
an intermediate portion between the first and second ends; and
a gripping layer provided over at least a portion of the intermediate portion, said gripping layer formed of a second material softer than the first material.

13. The medical device of claim 12, wherein the first material comprises a polymer having a hardness of at least 80 durometers and the second material comprises an elastomer having a hardness of less than 80 durometers.

14. A method of manufacturing a medical device handle, said method comprising:
forming a handle of a first material, said handle extending along a length and having a proximal end configured to be held proximate an area of interest of a patient and a distal end configured to be remote from the area of interest, said handle having an intermediate portion between the proximal and distal ends; and
forming a second material onto a gripping layer over at least a portion of said intermediate portion, said gripping layer being positioned along said handle to be held by a user, said second material being made of a softer material than said first material.

15. The method of claim 14, wherein said first material has a hardness of at least 80 durometers and said second material has a hardness of less than 80 durometers.

16. The method of claim 14, wherein said second material has a hardness of between 60 and 80 durometers.

17. The method of claim 14, wherein said forming comprises injection molding.

18. The method of claim 14, wherein said forming comprises one of overmolding and comolding said gripping layer and said handle.

19. The method of claim 14, further comprising bonding said gripping layer to said handle.

20. The method of claim 14, further comprising contouring said handle such that said intermediate portion has a smaller outer perimeter than an outer perimeter of said handle at said proximal end.
